(19) United States
(12) Patent Application Publication
(10) Pub. No.: US 2009/0204008 A1
(43) Pub. Date: Aug. 13, 2009

(54) WHOLE BODY INFRARED THERMOGRAPHY SYSTEMS AND METHODS

(76) Inventor: Daniel Beilin, Aptos, CA (US)

Correspondence Address: PILLSBURY WINTHROP SHAW PITTMAN LLP, P.O. BOX 10500, MCLEAN, VA, 22102, US

(21) Appl. No.: 12/028,743

(22) Filed: Feb. 8, 2008

(51) Int. Cl. A61B 5/01

(52) U.S. Cl. 600/474

(57) ABSTRACT

Systems, methods and apparatus are described for the collection and analysis of thermographic data. An infrared sensor is described that is mounted at an end of a probe and, together with a filter that restricts infrared radiation received by the infrared sensor to a selected band of wavelengths, a signal representative of a temperature at various points on the skin surface can be collected. Systems, methods and apparatus are described that facilitate the identification of target locations on the skin of a subject. Systems methods and apparatus are disclosed that can process a plurality of temperatures measured at different test sites on the skin surface and which provide information that includes an analysis of the plurality of temperatures.

### BACKGROUND OF THE INVENTION

1. Field of the Invention

The present invention relates generally to thermography and more particularly to medical infrared thermography.

2. Description of Related Art

Thermography is a used for measuring the amount of body heat delivered to the skin from a combination of cellular metabolism and the nervous system in targeted areas of the body. These measurements may be taken from a number of sites on the body. Conventional systems provide infrared camera systems that provide measurements for subsequent analysis of temperature patterns in order to attempt identification and/or a diagnosis of underlying medical conditions of the subject.

Conventional systems may also employ contact thermometry to obtain measurements. Contact thermometry relies on the conduction of heat between a detector and skin surface until the skin and the detector equilibrate at the same temperature, The direction of heat flow is generally from the warm skin surface to the colder detector. Therefore, measurement cools the skin and warms the detector until temperatures of the skin and detector reach a common intermediate between the two original temperatures. With a very small detector, the thermal mass or heat capacity of the object being measured (i.e. an area of the skin) will be considerably larger than that of the detector. Therefore the final, intermediate temperature will be near the initial temperature of the skin surface, within the measurement error.

The intermediate equilibrium temperature is reached after an interval of time related to the response time of the measuring instrument. A stable final temperature is said to have been measured when the change in the anticipated reading, if measurement is continued, is less than the measurement error. The response time depends on a number of different factors including thermal masses of the skin surface and of the detector, the temperature difference between skin surface and detector prior to the measurement, and the thermal conductance between skin surface and detector. The temperature-vs.-time function of the measurement is exponential; initially the temperature changes very rapidly, but the stable final temperature is reached very slowly and generally asymptotically.

Because the physical properties of the system cause the stable final temperature to be approached so slowly, it is usually impossible to specify the time required to obtain a reading. Instrument response times are therefore given as the time for the detector temperature to change by some fraction (e.g., 90%) of the difference between the initial temperature and the stable final temperature. Such response times are conventionally specified for conditions corresponding to the medical use of the instrument for surface temperature measurement and it has been the practice for manufacturers to-give response times based on trials with instruments immersed in water, and these times are considerably shorter. The only really useful data on response time are those obtained with a skin-surface phantom, which simulates the temperature of the skin surface.

There are a number of physical phenomena with characteristic temperature dependences that can be exploited as detectors in contact thermometers for skin-surface measurements. They include the following:

1. The volume or the pressure of solid, liquid or gaseous objects. The mercury clinical thermometer and bimetallic thermometers are classical examples of the application of temperature dependent materials.

2. The thermoelectric voltage difference at the point of contact of two different metals (Seebeck effect, thermocouple). The advantage of devices based on this principle is that the detector can be kept very small, so that its heat capacity is low and its response time very short. Moreover, when such thermocouples are connected in series to form a thermopile, very high temperature resolution can be achieved. Thermometry by this method encounters a technical difficulty in that the contact voltage of the measuring thermocouple must be compared with that of another thermocouple at a specific reference temperature. To establish a stable reference temperature is a technically elaborate procedure.

3. The electrical resistance or conductance of some semiconductors (thermistors). It is possible to produce thermistors that have specified properties and are artificially pre-aged, so that they are exchangeable and can qualify for official certification. Because of their small dimensions and hence low heat capacity, their response times can be made small.

4. The electrical resistance of most metals (e.g., platinum). The response time of a resistance thermometer can be made small by passing a high-frequency alternating current through the platinum wire, so that (by a “skin effect”) only the temperature change of a very thin outer layer of the wire is measured.

5. The resonant frequency of a quartz crystal. Instruments based on this principle can have high thermal resolution and remain stably adjusted for long times. But because of the very large thermal mass of the detectors so far available, their response times are very long. These detectors are at present unsuitable for the measurement of skin surface temperature.

However, reliability of contact thermography is limited because of numerous technical difficulties that can arise, including: inefficient heat conduction between detector and skin surface preventing the detector from reaching the same temperature as the skin quickly; pressure applied to the skin may altered the property to be measured; thermal mass of detector altering skin temperature; stability of ambient temperature; and interference with the mechanisms of heat transport between the skin and its surroundings.

### BRIEF SUMMARY OF THE INVENTION

These and other problems associated with conventional thermographic systems are addressed by certain aspects of the disclosed inventions. Certain embodiments of the invention provide systems, methods and apparatus that can be used for measuring skin temperature. An infrared sensor can be mounted at an end of a probe and a filter that restricts infrared radiation received by the infrared sensor to a selected band of wavelengths may be used in the generation of a signal representative of a temperature at the skin surface. Systems methods and apparatus are disclosed that can process a plurality of temperatures measured at different test sites on the skin surface and provide information that includes an analysis of the plurality of temperatures.

In certain of the disclosed embodiments, devices and methods are described that optimize efficiency and accuracy of thermographic systems. In one example, apparatus is used that maintains a desired separation between the infrared sensor and the skin surface.

Certain embodiments of the invention provide an infrared thermography systems, methods and apparatus that facilitate the identification of a target location on the skin of a subject. Information can be provided regarding the target location using a display of text and graphics as well as an audible signal. Location information may indicate the relationship of the target location to one or more landmarks. In some of these embodiments, providing location information includes displaying an image depicting the target location in relation to the one or more landmarks. In some of these embodiments, providing location information includes providing an audible description of the target location in relation to the one or more landmarks.

In certain embodiments, a probe may cooperate with one or more network devices in order to collect and analyze thermographic data. In certain embodiments, a process of collecting multiple readings from plural test sites enables rapid and accurate analysis of the collected data and facilitates diagnosis of underlying medical conditions.

### BRIEF DESCRIPTION OF THE DRAWINGS

FIG. 1 illustrates the use of a temperature probe according to certain aspects of the invention;

FIG. 2 is a simplified system diagram according to certain aspects of the invention;

FIGS. 3a and 3b depict an example of a probe; and

FIG. 4 is a block diagram showing functional elements found in certain embodiments of the invention.

### DETAILED DESCRIPTION OF THE INVENTION

Embodiments of the present invention will now be described in detail with reference to the drawings, which are provided as illustrative examples so as to enable those skilled in the art to practice the invention. Notably, the figures and examples below are not meant to limit the scope of the present invention to a single embodiment, but other embodiments are possible by way of interchange of some or all of the described or illustrated elements. Wherever convenient, the same reference numbers will be used throughout the drawings to refer to same or like parts. Where certain elements of these embodiments can be partially or fully implemented using known components, only those portions of such known components that are necessary for an understanding of the present invention will be described, and detailed descriptions of other portions of such known components will be omitted so as not to obscure the invention. In the present specification, an embodiment showing a singular component should not be considered limiting; rather, the invention is intended to encompass other embodiments including a plurality of the same component, and vice-versa, unless explicitly stated otherwise herein. Moreover, applicants do not intend for any term in the specification or claims to be ascribed an uncommon or special meaning unless explicitly set forth as such. Further, the present invention encompasses present and future known equivalents to the components referred to herein by way of illustration.

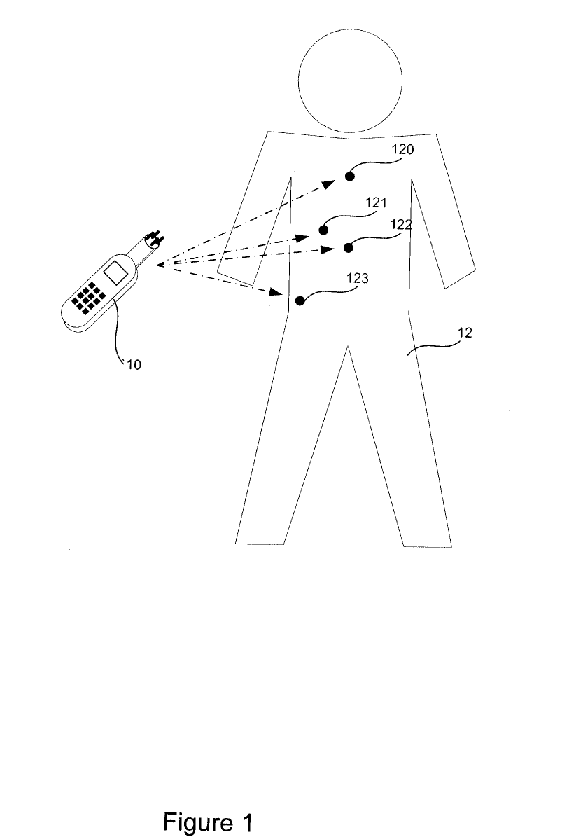

Certain embodiments of the invention provide systems and methods applicable in the practice of whole body thermography. With reference to FIG. 1, an infrared thermography device 10 can be used in the analysis of sequential changes in skin temperature at selected predetermined test sites 120-123 on a body 12. The predetermined test sites 120-123 can number in excess of 100 sites and typically exceed 120 test sites for a complete scan. In certain embodiments, the measured changes in skin temperature may be induced by stimuli that include variation of temperature, application of a noxious frequency and other stress stimuli. Variation in temperature can be accomplished by altering an ambient temperature, moving a subject from one enclosed area to another area having a different temperature and/or by removal of a covering of the subject body. In one example, after a series of temperature readings is taken while the subject is fully clothed and/or covered, a second series of readings can be taken after the subject is disrobed or otherwise uncovered; a delay between uncovering and taking the second readings may be introduced as desired or necessary. In the latter example, the skin surface of the subject may be maintained at a significantly higher temperature than the ambient temperature of the room in which measurements are taken. Patterns in the temperature changes at the predetermined test sites 120-123 can be used for diagnosis of underlying medical conditions.

Certain embodiments support analyses of thermodynamic temperatures and changes in thermodynamic temperatures. Thermodynamic temperature is independent of the nature of a thermometric substance and the properties associated with a particular substance and is formulated in terms of the position of a fixed reference point and the size of the base unit. In the international standard temperature scale, the base unit is the fraction 1/273.16 of the thermodynamic temperature of the triple point of water, and the fixed reference point (zero Kelvin) is absolute zero. The base unit is 1 Kelvin (1 K). Temperature is also measured in degrees Celsius (° C.). In the Celsius scale the base unit is the same as in the Kelvin scale, but absolute zero is at −273.16° C.

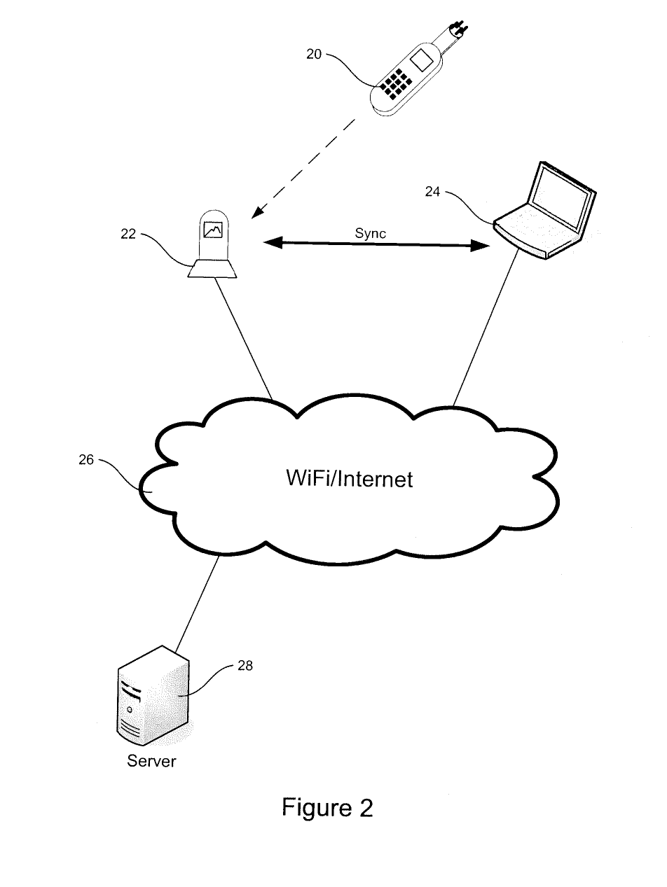

With reference to FIG. 2 certain embodiments of the invention comprise a base station 22 that supports operation of a temperature measuring probe 20. Probe 20 is typically operated by an operator or user trained to obtain temperature readings from predetermined test sites on a subject body. Base station 22 may provide a plurality of support services and functions for probe 20 and for the operator or user. In certain embodiments, base station 22 can receive temperature measurements obtained by probe 20 and can process the temperature readings before transmitting the processed results to a computing system 24. Probe 20 typically includes some processing capability as will be described in more detail below. Base station 22 may provide supplemental processing as well as data collection services to enable efficient communication between the probe 20, the base station 22, local computer 24 and one or more servers or data processing systems 26.

In certain embodiments, base station 22 and probe 20 may communicate using wired or wireless connections. In one example, data may be transferred from probe 20 using Bluetooth, WiFi or any suitable standard or proprietary wireless technology. In certain embodiments, probe 20 and base station 22 may be physically connected or physically connectable. In one example, a connecting cable may facilitate communication between probe 20 and base station 22. In another example, probe 20 may be docked with base station 22 enabling hard-wired communication between base station 22 and probe 20 and facilitating charging of batteries in probe 20 while the probe 20 is held in place by base station 22. Communication between base station 22 and probe 20 while docked or connected by cable may conform to a recognized commercial standard such as universal serial bus and/or to any other suitable standards-defined or proprietary communication protocol. While docked or connected by cable, probe 20 may exchange configuration information, software updates and measurement related data with the base station 22 and the local computer 24 or networked server 28. It will be appreciated that such information and data may just as well be exchanged wirelessly.

In certain embodiments, probe 20 and base station 22 may selectively communicate using a combination of wireless and wired communications methods where the communication method is selected based on factors including operator choice, location, ambient electromagnetic interference levels and regulations. In a medical facility, it may be necessary or required to curtail wireless communications in proximity to equipment that could be affected by wireless communications or that could create hostile environments for wireless communications. For example, communication between probe 20 and base station 22 may be restricted to wired communication where wireless communications used by the system might interfere with the operation of other patient monitoring equipment. In another example, proximity to electromagnetically noisy equipment may prevent secured reliable wireless communications between probe 20 and base station 22.

In certain embodiments, probe 20 and base station 22 may perform combinations of processes that enable a richer set of functionalities. For example, base station 22 may provide additional storage or buffers for data collected by probe 20. Furthermore, base station 22 may maintain configuration information and histories of prior readings obtained by probe 20. In certain embodiments, analysis of results may be performed by a combination of probe 20 and base station 22 although many embodiments provide a probe 20 that is capable of producing at least an initial analysis of results obtained by probe 20. In certain embodiments, probe may communicate directly with a local computer 24 or network server 28 and a base station 22 may be unnecessary for operation of the probe 20.

In certain embodiments, the primary functions of probe 20 include obtaining measurements of skin temperature of a subject at various test sites on subject's body. Measurements can be obtained as absolute temperature readings and may be expressed in degrees of Kelvin, Celsius or Fahrenheit as desired or selected by an operator. Measurements can also be obtained and expressed as a difference in temperature stated in degrees of Kelvin, Celsius or Fahrenheit. Difference readings may include one or more types of difference including a difference in successive readings at a single test site, a difference in readings between neighboring test sites, a difference between temperature at a test site and ambient temperature, a difference in measured temperature from a baseline temperature and so on. Probe 20 may record a combination of difference temperatures and absolute temperatures. In one example, probe 20 may record ambient temperature as an absolute reading and temperatures at a test site as variances from ambient temperature.

In certain embodiments, base station 22 receives temperature measurements obtained at a number of different test sites. Test sites may be grouped based on relationships that exist between group members and/or based on relevance of the group members to an analysis protocol and, potentially based on relevance of members to identification of one or more medical conditions. Groups may also comprise test sites that are to be measured in a desired sequence and/or timeframe. Temperature measurements may include a sequence of measurements obtained over a time period at a set of test sites. For example, the sequence may include two or more sets of readings, each set taken at different times. Analysis of the results may include a consideration of changes in conditions associated with the temperature measurements. For example, the subject from which measurements are taken may be located in a room in which ambient temperature is controlled to create stimuli that potentially affect skin temperature readings. In that regard, ambient temperature of the room may be adjusted after a first set of readings is obtained at a baseline ambient temperature. Adjustments to temperature may be made at a rate calculated to stimulate a desired response in the subject and, after a predetermined interval of time, a second set of readings may be obtained at the second ambient temperature. Analysis of results can include receiving information identifying the time and rate of change of ambient temperature. In certain embodiments, other stress stimuli may be applied that are calculated to obtain changes in body temperature. Application, nature and characteristics of a stimulus applied in this manner may be provided to the probe 20 for recording with associated measurements and analysis of results.

In certain embodiments, temperature measurements obtained from a plurality of test sites are analyzed by the probe 20 and, in at least some embodiments, by the base station 22. Typically, results are analyzed using pattern recognition techniques that may be embodied in computer programs and configured to identify one or more patterns in the collected results. Patterns may be detected within a selected set of test sites and may be further detectable between sets of measurements captured at different times. Consequently, patterns may be multidimensional in character having spatial and temporal components. In one example, temperature contours are mapped to a set of test sites and can then be used to identify temperature gradients on the skin of a subject. Additional sets of measurements can be obtained after a selected delay, and differences in the contours obtained from the successive sets of measurements can be mapped between points in time. As noted above, the differences may be induced by introduction of a stimulus to the subject between capture of the sets of measurements.

Pattern recognition techniques can be used to identify correlation between sets of results and reference patterns. The reference patterns may represent normal and/or ideal states. In certain embodiments, some reference patterns may represent abnormal states. Pattern matching may also be used to detect deviations from reference states. Thus, it may be possible to identify the onset of a medical condition or absence of medical condition by observing differences between measured results and results representing an ideal state. These differences may be monitored over a series of thermographic scans to identify trends away from normalcy and, in some instances, toward a known abnormal state. Additionally, it may be possible to detect trends towards normalcy when a subject had previously been associated with an abnormal or atypical medical condition.

In certain embodiments, patterns are identified using pattern recognition techniques that compare newly acquired sets of measured temperatures to models comprising patterns associated with certain medical conditions. Model patterns relevant to the current subject can be stored on the probe prior to measurement to facilitate real-time analysis. Testing may involve only a subset of the possible test sites of a subject. For example, the number of potential test sites may exceed 120 but certain test protocols may specify that only a portion of the test sites need be monitored during testing for a particular medical condition. A full scan of all test sites can be performed at any point in the test procedure to provide a spatial baseline of the subject, whereas other scans may be limited to one or more groups of test sites or region of the subject's body. Furthermore, the number of different groups and their constituents can be altered progressively or otherwise as a test protocol progresses.

In certain embodiments, the probe 20 may access additional model patterns available through the base station 22 and/or maintained by a local computer 24 or server 28 accessible through network 26. Model patterns may include a set of normal patterns representative of healthy subjects and deviations from the normal patterns that are indicative of a condition of interest. Patterns maintained by base station 22 or server 28 may include patterns associated with inflammation, disease, deficiency and various dysfunctions. For example, certain patterns of temperatures or temperature variations may be associated with diseases such as diabetes and cardiovascular disorders while other patterns may correspond with enzyme deficiencies and/or cognitive disorders. More than one pattern may be detected in a set of measurements and certain embodiments can correlate conditions with the presence of a plurality of detected patterns or known diseases.

In certain embodiments, results obtained for a subject may be stored in the probe 20 for future comparison. Typically, results are also transferred to the base station and to a host computer 24 and/or server 28. Results intended for storage may be communicated using wireless or wired communications and may include transmitting the results over a network 26 such as the Internet. Results that are stored may include raw data comprising temperature measurements obtained by probe 20, ambient conditions including temperature, rate of change of temperature, air pressure, humidity, etc. and characteristics of stimuli applied to the subject. Raw data may be further processed by a central system using the same or different pattern recognition tools. Furthermore, additional analysis of the raw data may be performed if new algorithms and/or reference patterns become available. Raw data and analytical results may be used to augment or modify a model or other reference patterns. In one example, the stored information of a subject who develops an infirmity or medical condition subsequent to testing may be evaluated with other the information of other patients in order to develop new or different reference patterns.

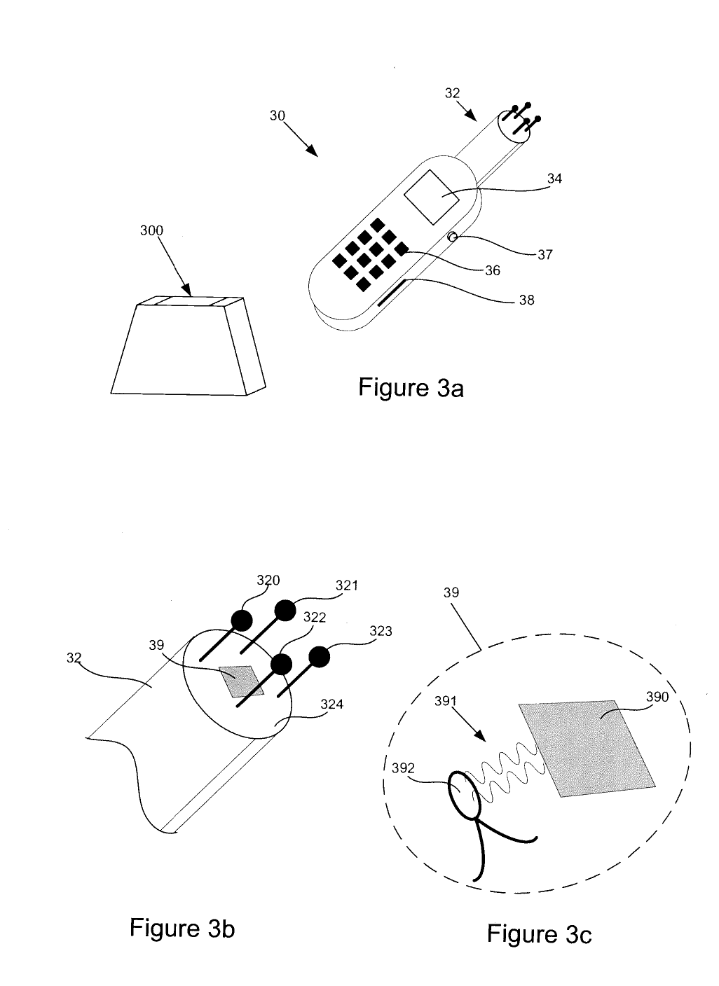

Referring now to FIG. 3a, a probe 30 may include an embedded computing system comprising a processor (not shown), a display 34, a communications interface provided internally or as a plug-in 38, one or more user input device 36, 37 and instructions for performing one or more pattern recognition algorithms. The processor may include one or more processing devices such as microprocessors, digital signal processors (“DSP”), custom logic arrays or other microcontroller. Communications interface 38 may be provided internal to probe 30 or may be provided as a plug-in component. A display 34 may be provided to provide system status, results including graphical displays and other information to an operator of the system. Input devices can include a keyboard/keypad 36, pushbutton 37, an optical reader, a microphone, etc. Typically, an operating system, measurement module, analysis tools and communications manager are provided to control temperature measurement and analysis of results. In certain embodiments, a real-time operating system is employed to provide accurate timing for measurement purposes.

In certain embodiments, a probe can be provided with instructions for conducting a testing procedure or protocol. For example, testing sites may be identified based on factors that include patient history and one or more targeted conditions. Thus, medical conditions associated uniquely with male patients may indicate a test procedure different from procedures for female patients. Similarly, certain medical conditions may have limited or no correlation with measurements observable at certain test sites. In certain embodiments, the test procedure can be conveyed to a user of the probe by means of an integral display. In some embodiments, an operator is prompted to obtain measurements from a next test site through a combination of textual description graphics and other signals. Locations for testing may be described textually in terms that describe a distance and direction (i.e. a vector) from known anatomical landmarks. In certain embodiments, an image identifying the site and/or its relationship to one or more anatomical landmark may be presented. In at least some embodiments, location information is provided audibly, typically through a wireless earphone or headset. In some embodiments, location information can be visually provided using an on-screen image. Location information can be provided in a plurality of communication methods. For example, a textual and/or verbal recitation of a landmark, e.g. “intercostal-2” can be accompanied with a display of a visual image or series of images that graphically shows the location of intercostal-2 on a body. In this manner, the probe can assist in training new operators.

In certain embodiments, upon locating the test site, an operator can capture a temperature by indicating that the probe is positioned for a reading in a manner discussed below in more detail. Upon measuring temperature at the site, the probe typically provides a signal to move to the next test site. The signal may be audible and/or visual and may include information identifying the next test site for measurement. In certain embodiments, the probe may present the operator with an option to navigate backwards to a previous test site or skip the current test site. In that regard, the probe may also identify questionable measurements that should be retaken or verified. Measurements may be questionable if they fall outside a reasonable range, are identified as problematic by an operator or are otherwise inconsistent with expected results. In a simple example, a reading of skin temperature of 26° C. or 38° C. may be considered questionable. The range of expected or valid temperatures can be configured based on prior results and/or measurements taken at neighboring test sites.

Typically probe 30 is in wireless communication with a base station 300 or a computing system (not shown). Base station 300 can be provided to charge batteries of the probe and to support processors for manipulating data obtained by the probe 30. For example, in certain applications, it may be desirable to minimize power consumption of the probe 30 while providing rapid processing of measurements obtained by probe 30. In some embodiments, measurement obtained by probe 30 can be relayed at the first opportunity to a base station 300 for processing and results of the processing can be returned by the base station for display on the probe. In the event that certain measurements appear to be outside of certain ranges, the base station 300 may signal an operator to take repeat measurements at one or more sites. Signaling can be accomplished using the display and other output capabilities of the probe 30, such as audible signals including synthetic and/or prerecorded spoken instructions.

In certain embodiments, probe 30 is provided with sufficient storage and processing power to process measurements without communicating with the base station 300. For example, probe 30 may be preloaded with past histories of measurements taken from a subject to be measured. In addition, the probe 30 may maintain sufficient information in storage to permit recognition of patterns in measurements acquired from a scan of a subject. For example, a probe 30 may support sufficient memory to maintain patterns for plural medical conditions as well as instructions and parameters that cause one or more processors in the probe to perform a variety of pattern matching techniques on measured data. Information stored in the probe 30 may include information that guides processing on measurements in a general case as well as specific selections and sequences of processes to be performed for the individual subject, a group of subjects and/or a class of subject. For example, a group of subjects may include individuals identified as having indicators indicating elevated risk factors associated with a particular medical condition. A class of subjects may include male and female classes, pediatric and adult classes, etc. In certain embodiments, probe 30 maintains raw data and processed results in storage. Storage may be provided internally and/or on removable media such as a memory card or smart card connected through a card slot 38. Where removable media is used, the removable media may be used as transport mechanism, a records management device and/or an archiving system.

Certain aspects of probe 30 will now be described with particular reference to FIGS. 3b and 3c. In certain embodiments of the invention, probe 30 measures heat using one or more infra red sensors 39. In the depicted example, an infrared sensor 39 is arranged on a surface 324 on the end of column 32 of probe 30. Members or probe tips 320-323 are provided on surface 324 and may perform various roles. For example, probe tips 320-323 may be formed as rods having ends located at a desired distance from surface 324 in order to obtain a desired separation of sensor from surface to be measured. Providing a consistent separation can improve accuracy and reproducibility of measurement. Furthermore, surface 324 may be provided as a generally flat, concave or convex profile as needed to maximize the efficiency of the infrared sensor. In some embodiments, ridges, undulations and other textures may be provided on surface 324 to increase available surface area for deploying an infrared sensor.

In certain embodiments, and as shown in FIG. 3c, infrared sensor 39 can comprise heat transducers 392 and filters 390. Suitable heat transducers 392 can include thermocouples, thermistors, charge coupled devices, photovoltaic devices sensitive to infrared wavelengths, infrared imagers and so on. In one example a thermocouple 392 is mounted in a cavity. Cavity can be lined with heat absorbing material such that the thermocouple 392 can measure heat induced in the lining. Cavity can be parabolic in shape and line with a reflective material such that a thermocouple 392 or other heat transducer may be located at a focus or at a focal plane of the thus formed parabolic mirror such that the heat transducer 392 receives a substantial majority of the heat energy 391 incident on the sensor 39 and passing through filter 390.

Filters 390 are employed in certain embodiments of the invention to restrict temperature measurement to measurement of heat energy 391 found at certain electromagnetic wavelengths. Typically, heat energy detected as infrared wavelengths in the 2-20 μm range provides information useful in certain thermographical applications. In particular, the 2-20 μm range of wavelengths excludes heat energy that is absorbed (and re-radiated) by water. Consequently, filtering infrared energy incident on the sensor 39 can produce information that may be attributed to activities and conditions of tissues, organs and systems of the subject body. Different applications may be better supported with filters that have narrower bandwidths. In one example, infrared wavelengths in range 2-14 μm are measured. In another example, an 8-12 μm filter can produce measurements that are of particular interest in regard to certain medical conditions. In certain embodiments, filter 390 comprises a Germanium crystal. Other filters may also be used. As necessary, two or more filters may be used to obtain a desired response to incident light. For example, in a two filter system, one filter may exclude light other than infrared wavelengths 8-20 μm while the second filter may pass only wavelengths in the band 2-12 μm; the resultant filter passes light in the 8-12 μm band.

In certain embodiments, filters may be interchangeable, configurable and/or tunable to permit testing of bands of infrared wavelengths. The bands may be narrow in relation to the infrared wavelength range of the probe. For example, in an embodiment where a probe can measure temperatures using any wavelength in the range 2-20 μm, a first filter may be used that passes only infrared radiation in the 4-8 μm band while a second filter may be employed for a second set of measurements using the 10-12 μm band. Differences in readings between the two bands may yield significant information related to the source of the infrared radiation. It is contemplated that bands spanning 4 or 5 μm of wavelength (e.g. 12-17 μm) and bands spanning significantly narrower bands (e.g. 9.8-10 μcm) may find application in certain applications. In particular, it is contemplated that an ability to discriminate between individual wavelengths or bands of wavelengths can yield significant useful information regarding underlying conditions of a subject.

In certain embodiments, ends of probe tips 320-323 can be electrically conductive such that probe 30 may include a voltage or current source that, when applied to the skin through the ends of probe tips 320-323 can be used to determine a measurement of electrical conductivity of the skin. Thus, resistances and impedances may be measured between combinations of pins, including between pairs of pins and between one pin and a plurality of other pins. Characteristics of currents passed through the skin may be used to determine resistance, inductance and capacitance.

In certain embodiments, the detection of skin impedance can be used to enable or start a temperature reading. In some embodiments, adequate skin contact is determined when measured impedance of the probe tips 320-323 falls within a range consistent with skin impedance. Contact with the skin can then be automatically determined and a temperature reading acquired without operator intervention; a signal can then be sent to the operator to move to the next test site. The probe display 34 may identify the next site to be tested enabling rapid navigation and temperature measurement acquisition from identified test sites.

In certain embodiments, one or more of pins 320-323 may be configured to mechanically activate/deactivate corresponding switches upon contact with the skin. Thus, when the switch is activated, the measurement of temperature can be enabled or initiated. More than one switch may be employed and multiple switches may be used to ensure proper alignment of the probe with the skin surface. Additionally, proximity detection may be performed by the probe 30 using non-physical means. For example, an angled spot of light may be projected on the skin by an LED and detected by a suitably positioned detector. In some embodiments, temperature is recorded only at the instruction of an operator. To this end, one or more push buttons 37 may be located on the probe 30 to allow the operator to command the probe 30.

In certain embodiments, and as illustrated in the example of FIG. 3a, the tips of pins 320-323 are provided with generally spherical or ellipsoidal ends. In some embodiments, the tips may be pointed, flattened or shaped as desired or necessitated by the application and types of measurements to be obtained.

Certain embodiments of the invention can provide a cost-effective measurement system capable of temperature resolution that is better than 0.1° C. and rapid response times. In the example, depicted, the probe 30 may be consistently located at an optimum distance from the site to be measured. While the probe in the example is suited for point measurement of skin-surface temperature and for integrated measurement of small skin areas, other embodiments may provide a plurality of probes that can be contacted to plural test sites on the skin of a subject.

In certain embodiments, probe 30 includes an ambient temperature measurement capability. However, in many embodiments, the ambient temperature may be obtained from an external device that is less affected by body heat of the subject or operator of the system. Ambient temperature may be communicated to the probe using wireless communications facilities of the probe such that temperature measurements may be stored as a temperature pair including ambient and measured contact temperatures. Ambient temperature may be used for on-going calibration of the probe whereby ambient temperature may be measured using a temperature sensor identical to the sensor 39 of the probe 30 in order to accommodate and adapt for certain non-linear characteristics of the devices used in the sensor.

In certain embodiments, temperature measurements are gathered as part of a dynamic temperature study. A baseline temperature profile of a subject is typically obtained at a first temperature. Typically, the subject has been exposed to the first temperature for a time sufficient to stabilize skin surface temperature when the baseline is obtained. One or more stimuli may then be applied to cause the skin temperature to change. For example, ambient temperature may be lowered and one or more sets of subsequent skin temperature measurements are obtained after time intervals, typically determined by the parameters of the study. In certain embodiments, a second or later set of readings is obtained after a predetermined time period that permits the establishment of steady state conditions. However, temperatures may continue to settle or regress to base line levels. Consequently, it may beneficial to obtain subsequent temperature measurements over a relatively short period of time. Additionally, some test protocols may require that temperatures be acquired from certain test sites within a predetermined time interval, while changes are occurring.

Therefore, timed test procedures may be supported by a probe 30. The probe 30 can be equipped with temperature sensors that quickly converge on an accurate measurement of temperature. Additionally, the sensors may be controlled such that a final temperature may be accurately predicted before convergence is completed. In one example, in certain dynamic studies where it is of particular importance that response time be as short as possible, a processor can be used to calculate the stable final temperature based on the rate of change and elapsed time of the measurement, permitting determination of the stable final temperature more rapidly than could be reached by the detector.

Probe 30 may capture a plurality of temperature readings which can be analyzed to determine a steady state temperature. The plurality of readings may comprise 30 or more temperature readings as necessary to permit a statistical analysis to determine steady state temperature with a desired degree of accuracy. Statistical analysis may include determining an arithmetic mean or median value. Statistical methods may be used to determine a final steady state value from readings that indicate that temperature is changing. Such statistical methods can include pattern matching and/or trend analysis methods.

When a probe 30 is to be moved relatively quickly from site to site, the probe 30 may be configured to set maximum and/or minimum times between readings and can provide prompts and other signals to an operator accordingly. In certain embodiments, multi-sensor probes or probe systems may be used to capture a plurality of readings simultaneously. For example, a probe may be configured with multiple temperature sensors positioned and oriented to obtain a plurality of readings from sites arranged around a central point of interest. Specific readings can then be selected for analysis and, in at least some embodiments, temperatures at desired test sites may be approximated or interpolated from neighboring measured test sites.

In certain embodiments, the probe may be used in conjunction with a multiple site monitoring system. The latter system can typically provide temperature monitoring at predetermined test sites enabling real-time monitoring of the predetermined sites. Additionally, a hand-held probe can be used to capture measurements at additional sites. The additional readings can be adjusted based on trends detected at the predetermined sites for which real-time measurements are obtained.

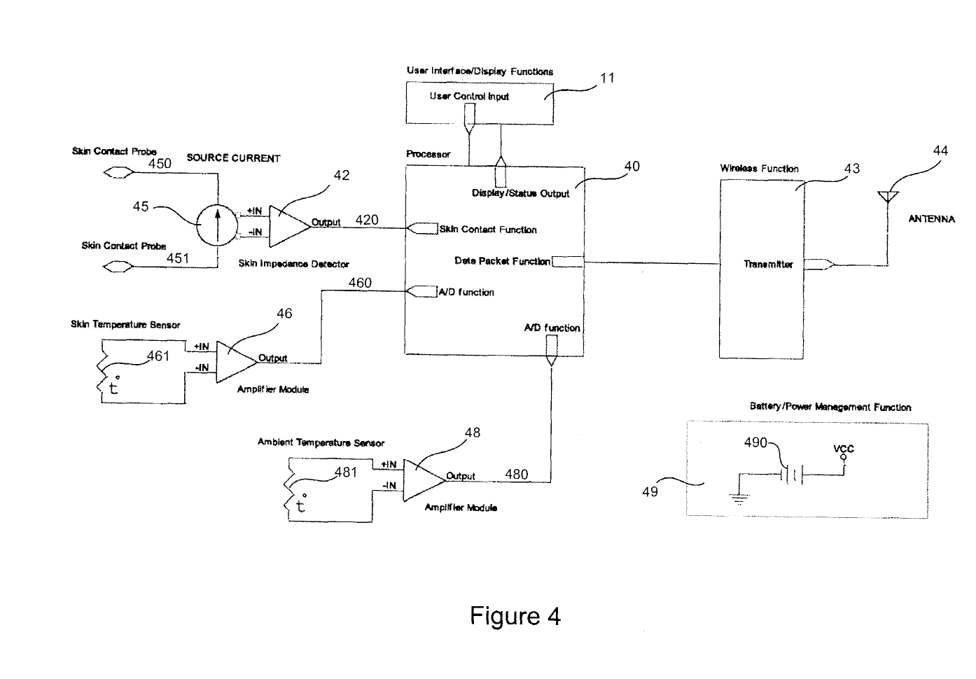

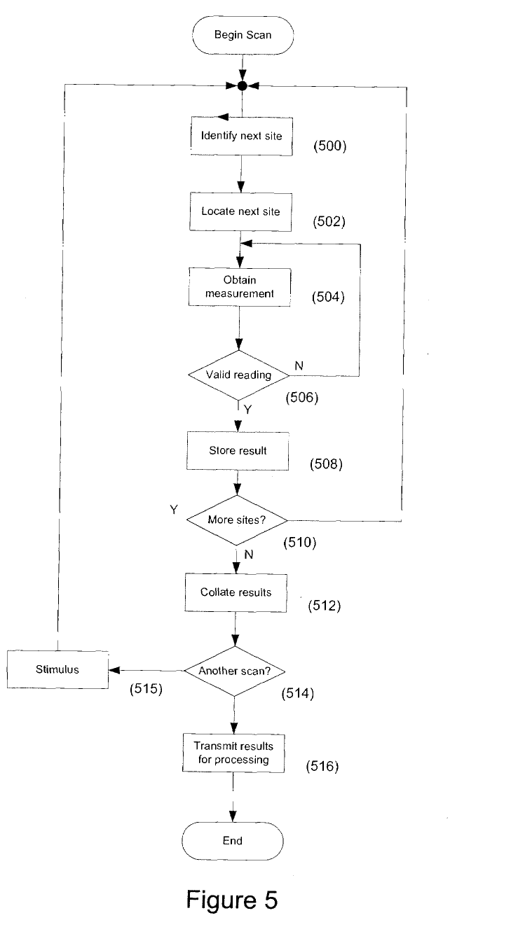

FIG. 4 includes a block diagram identifying elements of an example system provided according to certain aspects of the invention. Skin contact probes 450 and 451 provide a current from current source 45 to the subject skin. A comparator or voltage detector 42 measures voltage drop across current source 45 in order to provide a measurement 420 of skin impedance to processor 40. Skin temperature sensor 461 is monitored by voltage detector 46 which provides a signal 460 representing skin temperature to processor 40. Ambient temperature sensor 481 is monitored by voltage detector 48 which provides a signal 480 representing ambient temperature to processor 40. A user interface 11 is controlled by the processor 40, the user interface comprising switches, audio transducers including microphones, buzzers and loudspeakers, and display elements. Communications interface 43 facilitates communication between processor 40 and external devices, typically using wireless transmission through an antenna 44. Power management system 49 supports internal batteries 490 and external power supplies and controls charging of the batteries 490.

Turning now to FIG. 5, one example of a process for performing thermography is depicted. At step 500, a test site is identified at which a next temperature measurement should be taken. As described above, an operator maybe presented with a description or a graphic identifying the test site, typically in relation to an anatomical landmark. At step 502, the operator finds the test site and positions a probe adjacent to, or centered on the test site and at step 504, the temperature is measured. The temperature measurement may be triggered automatically based on location sensing devices on the probe and may also be indicated by activation of a button by the operator. When the probe has successfully obtained a temperature reading, the value may be checked for validity. To that end, the probe may maintain certain range information identifying maximum and minimum expected values of temperature. Validity may also be judged based on consistency of the reading with prior readings from the location or from neighboring locations. If the measurement is deemed invalid at step 506, a new measurement may be required, in which case the probe is typically removed from the subject skin and repositioned. In certain circumstances, the operator may be required to verify the location of the test site with regard to one or more anatomical landmarks.

When a valid temperature measurement has been obtained, the result is stored by the probe at step 508. The result may also be transmitted to a base station or workstation or network server. In certain embodiments, a display on the probe may present information derived from the measurement. For example, the value measured can be displayed along with other recent measurements at adjacent sites. In some instances, the result may be displayed together with previous or expected results for the site. If, at step 510, other sites are to be measured, steps 500-508 are repeated; otherwise, results may be collated at step 512. At step 512, results may be assembled into one or more sets of results that can be associated with points on the subject body, regions of the subject body and specific conditions. Collated sets of results can be stored locally as one of a series of scans and can be combined with other scans of the subject.

At step 514, it is determined whether the test protocol calls for another scan of the subject. Another scan may be performed for identical or different test points and may have some different and some common test points. Additionally, a next scan may be performed after introduction of a stimulus at step 515. Stimulus may include a heating or cooling of the ambient temperature or exposure to room temperature by removal of clothing or coverings. In certain embodiments, changes in skin temperature may be induced by altering an ambient temperature, moving a subject from one enclosed area to another area having a different temperature and/or by removal of a covering of the subject body. In one example, after a series of temperature readings is taken while the subject is fully clothed and/or covered, a second series of readings can be taken after the subject is disrobed or otherwise uncovered. Depending on the nature of the stimulus used and the type of analysis to be performed, a next scan may be performed after elapse of a predetermined time interval following the provision of the stimulus.

Results may be submitted for further processing at step 516. Processing may be performed for a complete set of results or by regions of the subject body. Results can be processed for a single test site or for a group of related test sites even if the sites in the group are not confined to a common region of the body. Results may be processed using previously obtained results. In certain embodiments, processing is performed locally within a combination of probe, base station and local computer. Additionally, in certain embodiments, further processing can be performed as a network service. In one example, results can be transferred to a network server for more detailed processing that may include advanced pattern recognition using a broader library of reference models and patterns. Network service may be provided to users on a subscription basis whereby a subscriber can request advanced or detailed processing according to agreed terms and conditions. Users may also submit results for processing subject to a per-use charge.

### Additional Descriptions of Certain Aspects of the Invention

Certain embodiments of the invention provide a probe for measuring skin temperature, comprising an infrared sensor mounted at an end of the probe, a filter that restricts infrared radiation received by the infrared sensor to a selected band of wavelengths, a processor for receiving from the sensor, a signal representative of a temperature at the skin surface and configured to process a plurality of temperatures measured at different test sites on the skin surface and a display providing information to an operator of the probe, the information including an analysis of the plurality of temperatures. In some of these embodiments, the filter comprises one or more Germanium crystals. In some of these embodiments, the filter is configurable to the selected band of wavelengths. In some of these embodiments, the filter is configurable by adding or removing certain of the Germanium crystals. In some of these embodiments, the filter is configurable by replacing the filter with a different filter having different optical properties. In some of these embodiments, the band of wavelengths comprises a portion of the wavelengths in the range 2-20 μm. In some of these embodiments, the band of wavelengths comprises wavelengths lying within 4 μm band. In some of these embodiments, the band of wavelengths comprises wavelengths lying within 2 μm band. Some of these embodiments further comprise one or more elongated members extending a predetermined distance from the end of the probe for maintaining a desired separation of the infrared sensor and the skin surface.

Certain embodiments of the invention provide an infrared thermography method comprising the steps of identifying a target location on the skin of a subject, providing location information to an operator of a thermographic probe, the location information indicating the relationship of the target location to one or more landmarks, upon receiving a positioning signal, measuring temperature of the target location using an infrared sensor in the thermographic probe, wherein the positioning signal indicates a predefined proximity to the skin at the target location, and selectively repeating the identifying, providing and measuring steps for a plurality of different target locations. In some of these embodiments, the step of measuring includes obtaining a plurality of temperature readings from the infrared sensor over a selected period of time and determining a steady state temperature reading for the target location from the plurality of temperature readings. In some of these embodiments, determining the steady state temperature includes performing a statistical analysis of the plurality of temperature readings. In some of these embodiments, the step of measuring includes receiving infrared radiation from the target location, wherein the infrared radiation is limited to a selected band of wavelengths. In some of these embodiments, the band of wavelengths is selected by one or more filters. In some of these embodiments, the one or more filters include at least one Germanium filter. In some of these embodiments, the step of measuring includes receiving infrared radiation from the target location, the infrared radiation being limited to a first band of wavelengths. Some of these embodiments further comprise selecting a second band of wavelengths and repeating the steps of identifying, providing and measuring for the second band of wavelengths. In some of these embodiments, at least some of the wavelengths are found in the first and second bands of wavelengths. In some of these embodiments, the first and second bands wavelengths comprise no common wavelengths. In some of these embodiments, the second band of wavelengths is narrower than the first band of wavelengths. In some of these embodiments, identifying the target location includes selecting a next target location from a set of locations on the skin of the subject, the set of locations providing information related to the medical condition of the subject. In some of these embodiments, providing location information includes displaying an image depicting the target location in relation to the one or more landmarks. In some of these embodiments, providing location information includes providing an audible description of the target location in relation to the one or more landmarks. Some of these embodiments further comprise comprising communicating the measured temperature to a networked device.

Certain embodiments of the invention provide an infrared thermography diagnostic system comprising a thermographic probe providing a plurality of temperature measurements obtained at a plurality locations on the skin of a subject, each temperature measurement obtained using a band-limited infrared sensor, a processor configured to process the plurality of skin temperature measurements to determine the existence of one or more patterns and a repository of pattern information, each pattern identifying an underlying medical condition, wherein the plurality of temperature measurements includes a first series of temperatures measured at a first ambient temperature and a second series of temperatures measured at a second ambient temperature, each measurement in the first series and a corresponding measurement in the second series being obtained at the same location on the skin. In some of these embodiments, the processor receives the plurality of measurements from the thermographic probe and performs one or more pattern matching methods on the plurality of measurements using the repository of pattern information from a network server. In some of these embodiments, the one or more pattern matching methods is based on a trend analysis of the plurality of measurements.

Although the present invention has been described with reference to specific exemplary embodiments, it will be evident to one of ordinary skill in the art that various modifications and changes may be made to these embodiments without departing from the broader spirit and scope of the invention. Accordingly, the specification and drawings are to be regarded in an illustrative rather than a restrictive sense.

What is claimed is:

1. A probe for measuring skin temperature, comprising:
an infrared sensor mounted at an end of the probe;
a filter that restricts infrared radiation received by the infrared sensor to a selected band of wavelengths;
a processor for receiving from the sensor, a signal representative of a temperature at the skin surface and configured to process a plurality of temperatures measured at different test sites on the skin surface; and
a display providing information to an operator of the probe, the information including an analysis of the plurality of temperatures.

2. The probe of claim 1, wherein the filter comprises one or more Germanium crystals.

3. The probe of claim 2, wherein the filter is configurable to the selected band of wavelengths.

4. The probe of claim 3, wherein the filter is configurable by adding or removing certain of the Germanium crystals.

5. The probe of claim 3, wherein the filter is configurable by replacing the filter with a different filter having different optical properties.

6. The probe of claim 1, wherein the band of wavelengths comprises a portion of the wavelengths in the range 2-20 μm.

7. The probe of claim 6, wherein the band of wavelengths comprises wavelengths lying within 4 μm band.

8. The probe of claim 6, wherein the band of wavelengths comprises wavelengths lying within 2 μm band.

9. The probe of claim 1 and further comprising one or more elongated members extending a predetermined distance from the end of the probe for maintaining a desired separation of the infrared sensor and the skin surface.

10. An infrared thermography method comprising the steps of
identifying a target location on the skin of a subject;
providing location information to an operator of a thermographic probe, the location information indicating the relationship of the target location to one or more landmarks;
upon receiving a positioning signal, measuring temperature of the target location using an infrared sensor in the thermographic probe, wherein the positioning signal indicates a predefined proximity to the skin at the target location; and
selectively repeating the identifying, providing and measuring steps for a plurality of different target locations.

11. The method of claim 10, wherein the step of measuring includes:
obtaining a plurality of temperature readings from the infrared sensor over a selected period of time; and
determining a steady state temperature reading for the target location from the plurality of temperature readings.

12. The method of claim 11, wherein determining the steady state temperature includes performing a statistical analysis of the plurality of temperature readings.

13. The method of claim 10, wherein the step of measuring includes receiving infrared radiation from the target location, wherein the infrared radiation is limited to a selected band of wavelengths.

14. The method of claim 13, wherein the band of wavelengths is selected by one or more filters.

15. The method of claim 14, wherein the one or more filters includes at least one Germanium filter.

16. The method of claim 11 wherein the step of measuring includes receiving infrared radiation from the target location, the infrared radiation being limited to a first band of wavelengths, and further comprising:
selecting a second band of wavelengths; and
repeating the steps of identifying, providing and measuring for the second band of wavelengths.

17. The method of claim 16, wherein at least some of the wavelengths are found in the first and second bands of wavelengths.

18. The method of claim 16, wherein the first and second bands comprise no common wavelengths.

19. The method of claim 16, wherein the second band of wavelengths is narrower than the first band of wavelengths.

20. The method of claim 10, wherein identifying the target location includes selecting a next target location from a set of locations on the skin of the subject, the set of locations providing information related to the medical condition of the subject.

21. The method of claim 10, wherein providing location information includes displaying an image depicting the target location in relation to the one or more landmarks.

22. The method of claim 10, wherein providing location information includes providing an audible description of the target location in relation to the one or more landmarks.

23. The method of claim 10 and further comprising communicating the measured temperature to a networked device.

24. An infrared thermography diagnostic system comprising:
a thermographic probe providing a plurality of temperature measurements obtained at a plurality locations on the skin of a subject, each temperature measurement obtained using a band-limited infrared sensor;
a processor configured to process the plurality of skin temperature measurements to determine the existence of one or more patterns; and
a repository of pattern information, each pattern identifying an underlying medical condition, wherein the plurality of temperature measurements includes a first series of temperatures measured at a first ambient temperature and a second series of temperatures measured at a second ambient temperature, each measurement in the first series and a corresponding measurement in the second series being obtained at the same location on the skin.

25. The system of claim 24 wherein the processor receives the plurality of measurements from the thermographic probe and performs one or more pattern matching methods on the plurality of measurements using the repository of pattern information from a network server.

26. The system of claim 25 wherein the one or more pattern matching methods is based on a trend analysis of the plurality of measurements.
